(19) United States
(12) Patent Application Publication
(10) Pub. No.: US 2024/0353513 A1
(43) Pub. Date: Oct. 24, 2024

(54) SYSTEM AND METHOD FOR NON-INVASIVELY PROBING IN-VIVO MITOCHONDRIAL FUNCTION USING FUNCTIONAL MRI (fMRI)

(71) Applicants: UNIVERSITY OF PITTSBURGH-OF THE COMMONWEALTH SYSTEM OF HIGHER EDUCATION, PITTSBURGH, PA (US); CEDARS-SINAI MEDICAL CENTER, LOS ANGELES, CA (US)

(72) Inventors: Yijen Wu, Wexford, PA (US); Anthony G. Christodoulou, Los Angeles, CA (US); Devin Raine Everaldo Cortes, Pittsburgh, PA (US)

(73) Assignees: UNIVERSITY OF PITTSBURGH-OF THE COMMONWEALTH SYSTEM OF HIGHER EDUCATION, PITTSBURGH, PA (US); CEDARS-SINAI MEDICAL CENTER, LOS ANGELES, CA (US)

(21) Appl. No.: 18/636,419

(22) Filed: Apr. 16, 2024

Related U.S. Application Data
(60) Provisional application No. 63/497,239, filed on Apr. 20, 2023.

(51) Int. Cl. G01R 33/48

(52) U.S. Cl. CPC G01R 33/4806; G01R 33/4818

(57) ABSTRACT

A method of non-invasively assessing mitochondrial function in live tissue of a subject includes providing periodic hypoxia challenges to the subject during a cyclic oxygenation period, acquiring 4D BOLD fMRI data from the live tissue of the subject during the cyclic oxygenation period, and analyzing the acquired 4D BOLD fMRI data to determine a measure of mitochondrial function for each of a number of regions of the live tissue. Also, a system for non-invasively assessing mitochondrial function includes an fMRI system including a magnet, an RF system and a controller, wherein the controller is structured and configured to acquire 4D BOLD fMRI data from the live tissue of the subject during a cyclic oxygenation period wherein periodic hypoxia challenges are experienced by the subject, and analyze the acquired 4D BOLD fMRI data to determine a measure of mitochondrial function for each of a number of regions of the live tissue.

### CROSS REFERENCE TO RELATED APPLICATIONS

This application claims priority to U.S. Provisional Patent Application Ser. No. 63/497,239, filed on Apr. 20, 2023 and titled “System and Method for Non-Invasively Probing In-Vivo Mitochondrial Function Using Functional MRI (fMRI)”, the disclosure of which is incorporated herein by reference.

### STATEMENT OF GOVERNMENT INTEREST

This invention was made with government support under grant #NS121706 awarded by the National Institutes of Health (NIH) and grant #W81XWH-22-1-0221 awarded by the US. Army Medical Research and Development Command (ARMY/MRDC). The government has certain rights in the invention.

### FIELD OF THE INVENTION

The disclosed concept pertains to the measurement and assessment of mitochondrial function in animal (e.g., human) tissue, such as the brain or heart, and, in particular, to a novel functional MRI (fMRI)-based system and method for non-invasively probing mitochondrial function in live tissue, such as the brain or heart, in a spatially specific manner.

### BACKGROUND OF THE INVENTION

Mitochondrial dysfunction is a critical element for wide ranges of inborn and acquired pathological conditions, such as mitochondrial respiratory chain disorders (MRCD), traumatic brain injury (TBI), sepsis, stroke, neurodegenerative diseases, childhood-onset epileptic encephalopathy, psychiatric disorders, and various heart diseases, especially heart failure with preserved ejection fraction. Living mitochondria taken from therapeutically removed brain tissues of TBI patients exhibited impaired oxidative phosphorylation function. Importantly, mitochondrial dysfunction is increasingly recognized as an inciting factor for acquired epilepsy after TBI. Therefore, mitochondria are emerging as promising therapeutic targets for brain injury and disorders. Neurons are highly sensitive to changes in oxygen and mitochondrial metabolism because oxidative phosphorylation, not glycolysis, powers presynaptic and postsynaptic activity, and broadly neurodevelopment, neurogenesis and synaptic plasticity. However, no non-invasive method is available that can probe in vivo mitochondrial functions in the intact brain in a spatially specific manner. Consequently, there is a lack of effective means for clinical diagnosis and surrogate endpoints for testing therapeutic efficacy.

Maintaining oxygen homeostasis is crucial for metazoan organisms like humans, exemplified by the 2019 Nobel Prize in Physiology or Medicine. To maintain oxygen homeostasis, acute hypoxia triggers fast cardiorespiratory reflexes with hyperventilation and sympathetic responses to increase pulmonary gas exchange and blood flow to bring more oxygen to vital organs such as brains and hearts. At the systemic level, the acute physiological response to hypoxia is triggered by oxygen-sensing systems, the arterial chemoreceptors in the carotid body, pulmonary arteries, ductus arteriosus, adrenal medulla or neuroepithelial bodies in the lung. The oxygen sensing by arterial chemoreceptors requires redox signaling of mitochondrial complex I in these peripheral oxygen-sensing systems. At the cellular level, oxygen sensing requires mitochondria, in particular respiratory chain activity and reactive oxygen species (ROS)-sensing mitochondrial signaling for transient compensatory activation of respiratory chain to maintain oxygen homeostasis and cellular metabolism. Both acute and chronic adaptation to hypoxia require mitochondrial functions. Mitochondria-controlled signaling is required for transient compensatory activation of the respiratory chain, a major mechanism of immediate adaptation to hypoxia, to maintain cellular oxygen homeostasis. Mitochondrial dysfunction therefore leads to an inability to accurately sense oxygen levels and maintain proper cellular/tissue oxygenation.

Blood-oxygen-level-dependent (BOLD) contrast has been used as a fMRI method to map human and animal brain activation and functional connectivity for decades, but has not previously been explored as a biomarker of mitochondrial function. BOLD MRI leverages the following two properties to map brain activation: (1) deoxy-hemoglobin is paramagnetic and can thus decrease MRI signals; and (2) neurovascular coupling response upon brain activation changes regional hemodynamics in the brain. Activated neurons need more glucose and oxygen supply. To meet the increased metabolic demands of activated neurons, the neurovascular coupling response in the brain increases the regional cerebral blood flow/volume to bring in more oxyhemoglobin for oxygen, which results in an increase in local MRI signals by changing the oxyhemoglobin/deoxyhemoglobin ratio. However, there are drawbacks to current fMRI approaches. There exists a temporal resolution, spatial resolution, and signal-to-noise ratio (SNR) trade-off in MRI. Static anatomical MRI is usually higher in resolution to better capture structural definition with no temporal information. On the other hand, BOLD fMRI, needing to capture fast dynamic information, is usually conducted with lower spatial resolution with larger voxel sizes and usually low SNR. It is a common practice to co-register the low-resolution fMRI maps onto high-resolution anatomical MRI images. This can result in incorrect mapping of functional activities onto anatomical structures. In addition, conventional BOLD fMRI is not feasible in the heart due to motion artifacts.

### SUMMARY OF THE INVENTION

In one embodiment, a method of non-invasively assessing mitochondrial function in live tissue of a subject is provided. The method includes providing periodic hypoxia challenges to the subject during a cyclic oxygenation period, acquiring 4D BOLD fMRI data from the live tissue of the subject during the cyclic oxygenation period, and analyzing the acquired 4D BOLD fMRI data to determine a measure of mitochondrial function for each of a number of regions of the live tissue.

In another embodiment, a system for non-invasively assessing mitochondrial function in live tissue is provided. The system includes an fMRI system including a magnet, an RF system and a controller, wherein the controller is structured and configured to acquire 4D BOLD fMRI data from the live tissue of the subject during a cyclic oxygenation period wherein periodic hypoxia challenges are experienced by the subject, and analyze the acquired 4D BOLD fMRI data to determine a measure of mitochondrial function for each of a number of regions of the live tissue.

### BRIEF DESCRIPTION OF THE DRAWINGS

A full understanding of the invention can be gained from the following description of the preferred embodiments when read in conjunction with the accompanying drawings in which:

FIG. 1 is a flowchart illustrating a method of 4D Oxy-wavelet MRI according to an exemplary embodiment of the disclosed concept;

FIG. 2 is a schematic diagram illustrating an exemplary implementation of a method of 4D Oxy-wavelet MRI according to an exemplary embodiment of the disclosed concept;

FIG. 3 is a flowchart illustrating a method of rapid BOLD-fMRI data acquisition according to an exemplary embodiment of the disclosed concept;

FIG. 4 is a flowchart illustrating a method of BOLD-fMRI waveform analysis according to an exemplary embodiment of the disclosed concept;

FIG. 5 shows example oxy-wavelet index maps for exemplary normal and abnormal tissue according to an exemplary embodiment of the disclosed concept; and

FIG. 6 is a schematic diagram of an fMRI system according to an exemplary embodiment in which the various embodiments of the methodology described herein for non-invasively probing mitochondrial functions in live tissue in a spatially specific manner may be implemented.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the term “system” is intended to refer to a computer related entity, either hardware, a combination of hardware and software, software, or software in execution. For example, a system can be, but is not limited to being, a process running on a processor, a processor, an object, an executable, a thread of execution, a program, and/or a computer. By way of illustration, both an application running on a server and the server can be a system. One or more systems can reside within a process and/or thread of execution, and a system can be localized on one computer and/or distributed between two or more computers.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the disclosed concept. It will be evident, however, that the disclosed concept can be practiced without these specific details without departing from the spirit and scope of the disclosed concept.

The disclosed concept provides a novel functional MRI (fMRI) methodology called “4D Oxy-wavelet MRI”, as a non-invasive biomarker of mitochondrial function underlying its ability to maintain oxygen homeostasis in vivo in a motion-resolved and spatially specific manner. A 4D (four dimensional) Oxy-wavelet MRI scan according to the disclosed concept simultaneously monitors oxygen homeostasis and structural properties in live tissue, such as in a beating heart or in a live brain, in a spatially specific manner with high-resolution. In addition, such a scan can be useful for detecting mitochondrial dysfunction in both rare genetic mitochondrial diseases, as well as in injury or diseases where mitochondrial dysfunction is secondary to the injury or genetic abnormality. Preliminary data relating to the disclosed concept has demonstrated its applicability to fetal mouse brains, adult mouse brains, and mouse hearts, indicating its translational utility for diagnosis not only in human children and adults, but also prenatally in developing fetuses.

As noted elsewhere herein, mitochondrial dysfunction leads to an inability to accurately sense oxygen levels and maintain proper cellular/tissue oxygenation. The 4D Oxy-wavelet MRI of the disclosed concept leverages this manifestation as a biomarker that can reflect mitochondrial dysfunction in, for example, the brain and heart. The 4D Oxy-wavelet MRI of the disclosed concept can simultaneously capture anatomical and functional features in the same single scan with both high-spatial and temporal resolution (e.g., frame rate: ˜14 msec) using a sub-Nyquist sparse sampling scheme. Since the functional signals are captured in the same anatomical scan, this allows capturing fast BOLD signal changes with both high spatial and temporal resolution and high SNR as anatomical images without the common pitfalls of fMRI. In addition, the novel 4D-fMRI scheme of the disclosed concept can model fast respiratory, cardiac, and fetal motions, and thus can perform 4D fetal cardiac imaging without motion artifacts. This enables free-breathing-no-gating 4D-fMRI for non-invasive imaging. This can facilitate patient imaging without breath holding and animal imaging with minimal anesthesia. In conjunction with atlas-based brain region parcellation, the 4D Oxy-wavelet MRI of the disclosed concept enables brain region-specific evaluation of mitochondrial dysfunction in vivo. As described in great detail herein, the disclosed concept uses time-frequency analysis of BOLD signals in response to oscillating hypoxia challenges to probe the integrity of mitochondrial function. The unique oxygen cycling waveform analysis enables artificial intelligence (AI)-based automatic diagnosis for future clinical applications.

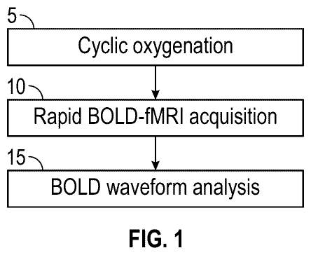

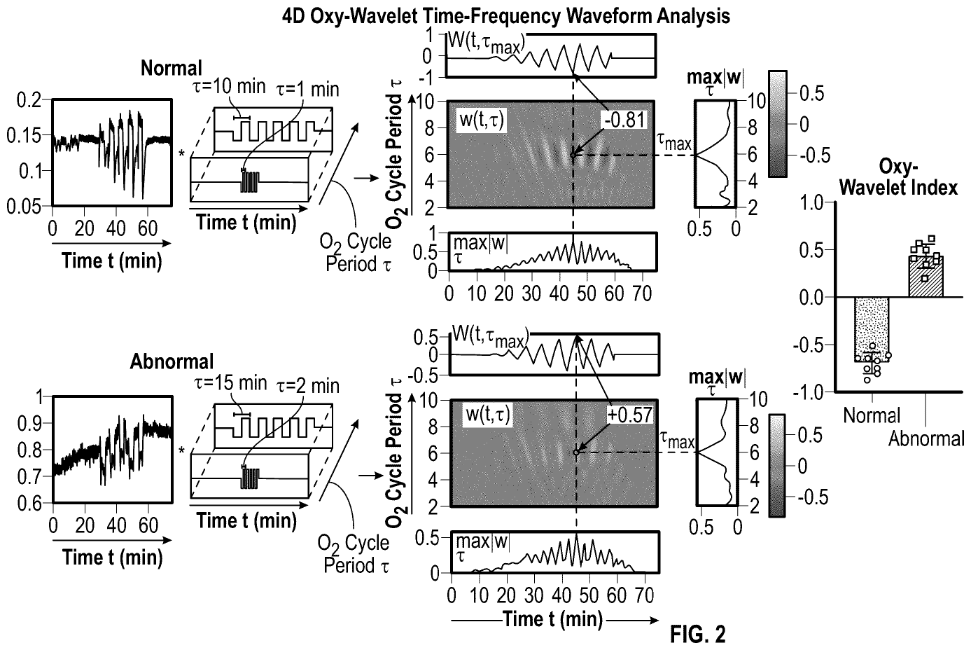

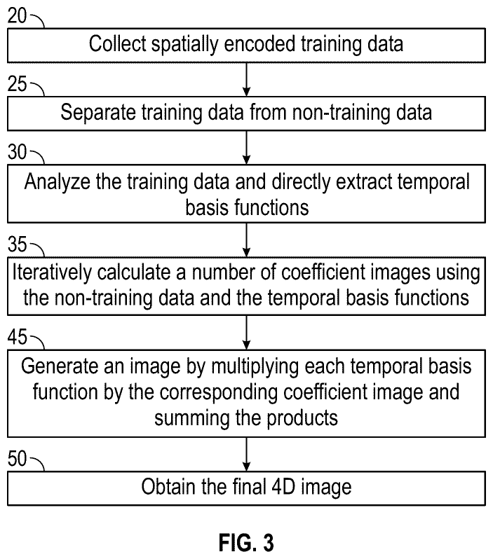

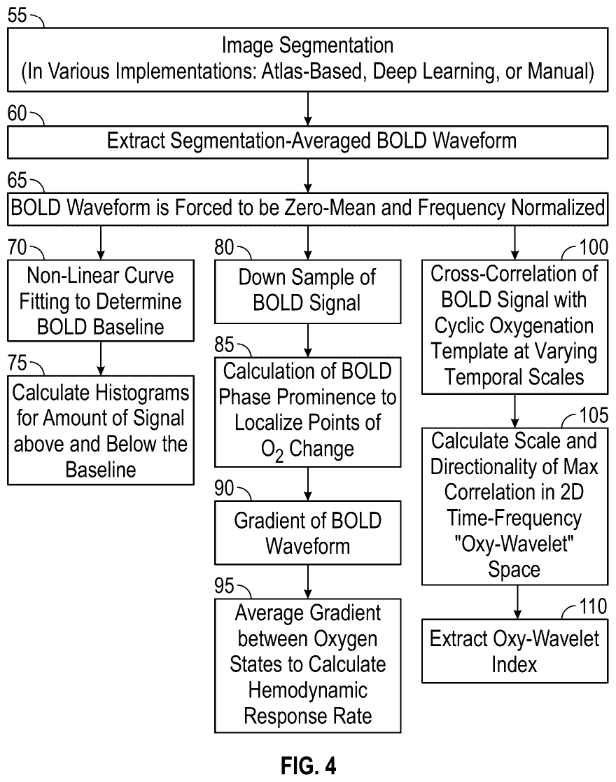

FIG. 1 is a flowchart illustrating a method of 4D Oxy-wavelet MRI according to an exemplary embodiment of the disclosed concept. As shown, the method includes three main components. The three main components are: (i) cyclic oxygenation inputs to the subject, shown in box/step 5, (ii) rapid 4D BOLD fMRI data acquisition during the cyclic oxygenation, shown in box/step 10, and (iii) BOLD waveform analysis of the acquired BOLD fMRI data, shown in box/step 15. “4D” refers to a time series of the 3D isotropic BOLD fMRI acquisition. Being “rapid” refers to the requirement of a high frame rate or temporal resolution to resolve the hemodynamic responses for the 3D time series in a voxel-wise manner. The temporal resolution for each 3D frame is typical in the 10s of msec range (e.g. ˜14 to 80 msec per 3D frame). Particular exemplary embodiments and implementations of each of these components are described in greater detail herein. Specifically, certain aspects of an exemplary implementation of the method of 4D Oxy-wavelet MRI are shown schematically in FIG. 2 and in flowcharts provided in FIGS. 3 and 4 (described in detail herein).

Referring again to FIG. 1, box/step 5 thereof includes the provision of cyclic oxygenation to the subject during data acquisition. In particular, the cyclic oxygenation of box/step 5 includes the oscillation of oxygen levels that are provided to the subject during the imaging study as described herein. The development of the disclosed concept was performed in connection with anesthetized animals (i.e., mice), wherein the cyclic oxygenation involved subjecting the anesthetized animals to rapid cycles of periodic hypoxia (e.g., 3-minute cycles of 10% O₂) challenges interspersed between cycles of hyperoxia (e.g., 3-minute cycles of 100% O₂). It is important to note that the exact oxygen levels (10%, 20%, 100%, etc.) and cycle periods are not critical to implementation of the disclosed concept, whether in human or non-human subjects. Rather, the important feature is that oscillation of oxygen levels is induced during the imaging study. For animal studies, acute 3-minute hypoxia (e.g., 10% O₂) challenges may be given through inhalation via a nose cone. In human subjects, acute hypoxia challenges may be performed through either a) controlled subject breath holding, or b) an oxygen tank and nasal cannula or the like coupled to the subject.

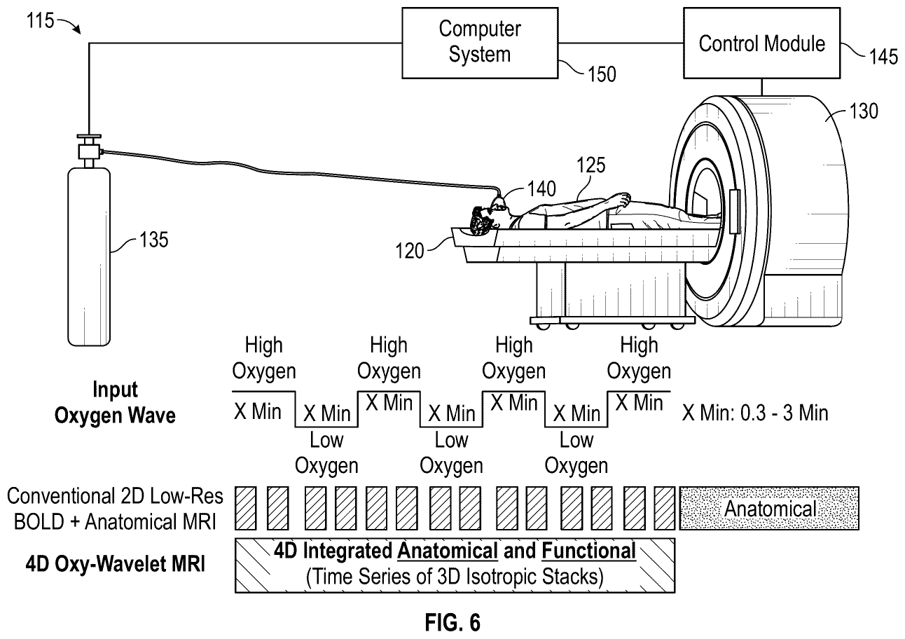

Box/step 10 shown in FIG. 1 relates to the data acquisition that is performed during the provision of the cyclic oxygenation to the subject as described above. In particular, while the cyclic oxygenation is provided top the subject, rapid 4D BOLD fMRI data is acquired using a suitable fMRI system. One such suitable fMRI system is described elsewhere herein in connection with FIG. 6.

In one non-limiting exemplary implementation of box/step 10, the rapid BOLD-fMRI data acquisition is performed according to a method that is shown in FIG. 3. Referring to boxes/steps 20 and 25 of FIG. 3, the method of this exemplary embodiment employs a data acquisition strategy which alternates between collecting two sets of BOLD fMRI data. The first set of data (D1), also referred to herein as training data, contains auxiliary “navigator” data collected at a high temporal sampling rate matching the target frame rate], but with k-space coverage that would not otherwise be sufficient to resolve spatial features (i.e., does not need to satisfy Nyquist requirements or maximum extent for the desired spatial coverage and resolution). In some implementations D1 constitutes repetition of a single unique k-space trajectory. The second set of data (D2), also referred to herein as non-training data, contains sparsely sampled (k, t)-space data with extended k-space coverage. It is sparsely sampled in the sense that it does not on its own satisfy both the Nyquist requirements and the temporal sampling rate associated with the desired spatiotemporal resolution. It has extended k-space coverage in the sense that it covers more of k-space than D1, and satisfies the maximum extend required for the desired spatial resolution. This strategy synergizes with subspace-based image models such as the partial separability model I(x, t)=Σₗ₌₁^{L}ψₗ(x), ϕₗ(t), where I(x, t) is a 4D image of space x and time t. In this context, the high-speed data in D1 informs temporal basis functions (i.e., the q's), and the full-k-space data in D2 informs spatial coefficient maps (i.e., the d's), resulting in images with the high temporal resolution of D1 and the high spatial resolution of D2.

Referring to box/step 30 of FIG. 3, the method proceeds by analyzing the training data (D1) and directly extracting therefrom a number of temporal basis functions. In the exemplary embodiment, the temporal basis functions can be taken from the singular value decomposition (SVD) of D1. Assumed temporal functions (e.g., constant waveform or waveforms matching or decomposing the oxygenation event) may be added as further φ's. The functions can form a matrix Φ with elements ϕᵢⱼ=φᵢ(t_{J}), which span a temporal subspace that will be used to constrain the image reconstruction of I(x, t). Next, at box/step 35, a number of coefficient images are iteratively calculated using the non-training data (D2) and the temporal basis functions extracted in box/step 30. In an exemplary embodiment, one way to use Φ to constrain reconstruction of I(x, t) is to directly determine the spatial coefficient maps Ψ with elements Ψᵢⱼ=ψⱼ(xᵢ) by regularized least-squares fitting of the temporal basis to the remainder of the imaging data. This step is calculated according to the following:

$$\\ Ψ = arg {min}_{Ψ} {{‖ d - E ( Ψ Φ ) ‖}_{}}_{2}^{2} + R ( Ψ ) ,$$

where d are the measured data, E is the encoding operator comprising spatial encoding and under-sampling, and R is a regularization functional on Ψ (e.g., a wavelet-spectral sparsity penalty). To overcome motion artifacts and remove noise, both low-rank imaging (subspace imaging) and compressed sensing (through R(⋅)) are employed for removal of artifacts and de-noising. This approach exploits the correlation of images over time as well as the transform sparsity of the image series to allow image reconstruction from sparse samples. The reconstruction and compressed sensing orders and parameters can be optimized to increase signal to noise ratio (SNR) and motion blurring for 4D whole-volume motion-resolved fetal MRI. Commonly used fast imaging schemes, such as echo-planar imaging (EPI) with spiral k-space sampling, suffer from motion artifacts for mouse pregnancy with multiple fetuses with multiple beating fetal hearts and movements. On the contrary, the hybrid low-rank strategy of the disclosed concept can image multi-fetal mouse pregnancy without motion artifacts and with both high spatial [(78 μm)³-(120 μm)³] and high temporal (˜14 msec) resolutions.

After box/step 35, the method proceeds to boxes/steps 45 and 50, wherein a 4D image is obtained and finalized by multiplying each temporal basis function by the corresponding coefficient image and summing the products. The 4D image can be seen as a “stack” of 3D images, where each image in the stack is from a different time point of the process. By combining D1 temporal functions and D2 basis functions in the manner described, and by exploiting the fact that each frame in the fetal MRI time series has a high degree of spatiotemporal correlation (i.e., they reside in a low-dimensional subspace), the method of the disclosed concept is able to reduce the degrees of freedom required to represent a sequence of images. The method of the disclosed concept can thus achieve imaging speeds beyond those which parallel imaging or fast scanning can offer.

In one non-limiting exemplary implementation of box/step 15, the BOLD-fMRI waveform analysis may be performed according to a method that is shown in FIG. 4. Referring to box/step 55 of FIG. 4, the method beings by segmenting the final 4D image that was constructed in box/step 10 of FIG. 1 and, in the exemplary embodiment, in box/step 50 of FIG. 3. The particulars of the image segmentation of box/step 55 can vary depending on the biological system being imaged.

In one non-limiting exemplary embodiment, for adult animal brain imaging, the image segmentation of box/step 55 may be performed according to the following pipeline. In short, the final 4D image is rigidly registered to an atlas space suitable for the imaging object. For example, for mice, the 4D images are the Allen brain template (Allen Institute for Brain Science, USA) and are segmented into gray matter (GM), white matter (WM) and cerebrospinal fluid (CSF) tissue maps using the Unified Segmentation approach (Ashburner, J. and K. J. Friston, Unified segmentation. Neuroimage, 2005. 26 (3): p. 839-51); as implemented in the statistical parametric maps (SPM) (Friston, K. J., et al., Statistical parametric maps in functional imaging: A general linear approach. Human Brain Mapping, 1994. 2 (4): p. 189-210). For humans, a different atlas would be used. For the segmentation task, the tissue probability maps (TPMs) of Hikishima et al. (Hikishima, K., et al., In vivo microscopic voxel-based morphometry with a brain template to characterize strain-specific structures in the mouse brain. Sci Rep, 2017. 7(1): p. 85) served as priors for tissue classification. A weighted image is then constructed using the tissue segments of the animal and the TPMs of the template, respectively. The weighted images are co-registered using affine and nonlinear B-spline transformation via Elastix package (Klein, S., et al., elastix: a toolbox for intensity-based medical image registration. IEEE Trans Med Imaging, 2010. 29(1): p. 196-205). The resulting parameters for forward transformation were stored to allow a subsequent image transformation from native animal space to Allen mouse template space. Parameter files for inverse transformation were also generated and stored to allow a subsequent image transformation from template space to native space (e.g. hemispheric brain mask). Using the files for inverse transformation, the template is transformed to the native, 1^{st} volume of the data to create the final brain segmentation mask.

In another non-limiting exemplary embodiment, cardiac imaging, or fetal mouse scans, manual segmentation is performed, roughly following the below pipeline. First, a single, 3D isotropic anatomical image is acquired from the 4D image stack obtained in box/step 50. The 3D isotropic anatomical image is either a single timeframe from the 4D stack or an average of specific time indices. This image, either in Digital Imaging and Communications in Medicine (DICOM) or Neuroimaging Informatics Technology Initiative (NIfTI) format is loaded into the biomedical image viewer, ITK-Snap (http://www.itksnap.org/pmwiki/pmwiki.php). ITK-snap's built-in polygon drawing tool is then used to draw ROIs (region of interest) or VOIs (volume of interest), covering the biological tissue of choice. These segmentations are saved as a new file and used in downstream analysis.

In the future, for tissue that does not have current automated segmentation methods, the disclosed concept may employ deep learning into the pipeline. Generative Adversarial Networks (GANs) or Convolutional Neural Networks (CNNs) can be trained to perform automatic segmentation from the segmentation files generated in the previous paragraph.

Following box/step 55, the method proceeds to box/step 60, wherein the segmentation files generated in the previous description are used to extract tissue-specific BOLD information. The spatial coefficient maps and temporal basis functions described previously are multiplied and summed over the indices where the segmentation indicates there is tissue of interest. The result is a BOLD waveform stored as a vector for downstream analysis.

Next, at box/step 65, the BOLD waveform is forced to be zero-mean and frequency normalized. In particular, the mean of the tissue-specific BOLD signal is subtracted from each BOLD timepoint. The zero-mean signal is then frequency-normalized by dividing each signal by the RMS (Root-mean-square) value of the signal according to the following formula:

$$\\ RMS ⩵ \sqrt{\frac{1}{n} {\sum }_{i} {x}_{i}^{{}_{} 2}} ,$$

where xᵢ is a single time point of the bold waveform.

Following box/step 65, the method may proceed to any of or a combination of two or more of three possible further analysis scenarios. In particular, as seen in FIG. 4, the method may proceed to a first scenario including boxes/steps 70 and 75, a second scenario including boxes/steps 80, 85, 90 and 95, and/or a third scenario including boxes 100, 105, and 110. Each of these different scenarios is described in detail below.

The first scenario begins at box/step 70, wherein non-linear curve fitting is employed to determine a BOLD baseline. In particular, in the exemplary embodiment, an nth order polynomial is fit to the BOLD curve to account for linear and higher-order baseline drift that often appears in the BOLD signal. This polynomial is subtracted from the BOLD waveform to remove this drift. Then, at box/step 75, histograms are calculated for the amount of the signal that is above and below the baseline. Specifically, the BOLD waveform is split into individual oxygen-flipping waveforms. For example, if an animal undergoes 3 flips from 10-100%, 3 minutes per event, then the amount of BOLD waveform timepoints above and/or below the baseline during the 10% hypoxia challenge is calculated. This is done by comparing the change in BOLD during the 10% hypoxia to the BOLD signal during the 100% hyperoxia.

The second scenario begins at box/step 80, wherein the BOLD signal is down sampled. In particular, in the exemplary embodiment, the BOLD signal is down-sampled by a factor of 10 to remove-redundant sample points during oxygen-transitions that increase the noise when performing discrete-gradients in later steps. Discrete gradients are essentially derivatives of non-symbolic, non-continuous signals. The simplest form of a discrete gradient is simply the first finite-difference. Defined in words as subtract each point of the BOLD waveform by the point directly after it in time. This can be represented as the following equation:

$$\\ y [ n ] = x [ n ] - x [ n - 1 ] .$$

Then, at box/step 85, BOLD phase prominence is calculated to localize points of O₂ change. The BOLD signal is a complex signal, and thus has both phase and magnitude. The phase of the BOLD signal is used to determine the points with high likeliness to represent a change in oxygen states. The peak-prominence of the phase of the BOLD signal is used to determine the points most likely associated with changes in oxygen states. In words, topographical prominence is defined as the height of the BOLD signal compared to the lowest contour in. neighborhood around that point. Next, at box/step 90, the first gradient of the BOLD waveform is calculated. Then, at step 95, the gradient between oxygen states is averaged to calculate a hemodynamic response rate. More particularly, points identified in box/step 80 dictate ranges of BOLD timepoints where a change in oxygen state is occurring. The average of the BOLD gradient is calculated during the BOLD signal when a change from hypoxia to hyperoxia occurs. The average of the signal during this time point represents the average rate it takes the supplied oxygen to get to tissue. It is a representation of the hemodynamic response rate.

The third scenario includes boxes/steps 100-110. In step 100, the BOLD signal is cross correlated with the cyclic oxygenation template at varying temporal scales. In step 105, the scale and directionality of the max correlation in the 2D time-frequency “oxy-wavelet” space is calculated. Then, at step 110, an oxy-wavelet index is extracted.

More specifically, with reference to boxes/steps 100-110, cross-correlating the BOLD signal with template event waveforms across multiple oscillation frequencies can be interpreted as a variant of the Continuous Wavelet Transform (CWT) with the oxygen template event waveform serving as a modified mother wavelet. This approach generates a two-dimensional feature space that unveils various details, with one axis representing the correlation over distinct time shifts (a translation parameter ‘b’) and the other axis corresponding to the correlation across the oscillation frequencies (a scale parameter ‘a’).

The modified CWT-like procedure for cross-correlating the BOLD signal and the template oxygen event waveforms in the non-limiting exemplary embodiment is as follows: (1) shift (translate) the oxygen template event waveform over the preprocessed BOLD signal by varying the translation parameter ‘b’; (2) for each shift increment (time-shift), compute the product of the BOLD signal and the time-shifted oxygen event waveform, and store the sum of the resulting values (akin to computing the CWT for the given ‘a’ and ‘b’); and (3) modify the oxygen oscillation frequency (scale) of the template by adjusting the scale parameter ‘a’, and repeat steps (1) and (2). The resulting information not only reveals the directionality of the correlation but also identifies the oxygen-frequency (corresponding to the scale parameter ‘a’) at which the maximum correlation occurs. In other words, the location of the peak-magnitude value reveals the midpoint of the oxygen cycling experiment and the oscillation period (neither of which is precisely known a priori when oxygen switching is manually triggered). More importantly, the sign of the peak value reveals the direction of oxygen response. If the processed BOLD waveform is negatively correlated with the oxygen template, this represents an active response to the hypoxia challenge and thus, intact mitochondrial function. If the processed BOLD waveform is positively correlated with the oxygen template, this represents a passive response to the hypoxia and indicates some form of mitochondrial dysfunction, dysregulation.

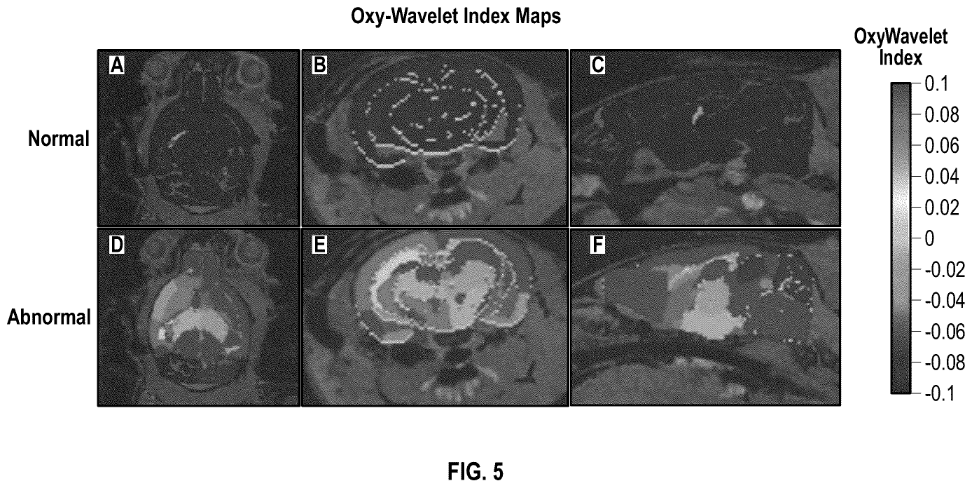

In one particular exemplary embodiment, the oxy-wavelet index that is determined for each of a number of ROIs of the tissue in question may be used to create a color-coded Oxy-wavelet index maps (also referred to as spatial maps). Example oxy-wavelet index maps for exemplary normal and abnormal tissue are shown in FIG. 5.

FIG. 6 is a schematic diagram of an fMRI system 115 according to an exemplary embodiment (which may be a full body scanner or, alternatively, a head only scanner) in which the various embodiments of the methodology described herein for non-invasively probing mitochondrial functions in live tissue in a spatially specific manner may be implemented. In particular, the image acquisition and analysis methods described herein, in the various embodiments, may be implemented as a number of software routines embedded in the control system of fMRI system 115.

Referring to FIG. 6, fMRI system 115 includes a table 120 on which a subject 125 rests. Table 120 is structured to slide inside a tunnel formed by a housing 130. Housing 130 houses a superconducting magnet, which generates a very high magnetic field. Housing 130 also houses a gradient coil that is integrated with the magnet for adjusting the magnetic field. Housing 130 further houses a Radio Frequency (RF) assembly or system, which applies RF pulses to a specific body-part of the subject 125 to be analyzed, and receives signals that are returned by the same body-part. The RF assembly or system may be, for example, a surface coil system, a saddle coil system, a Helmholtz coil system, an RF transceiver array system, or any other suitable RF system or structure. A magnetic shield is provided, which surrounds the magnet, gradient coil and RF system, and which minimizes the magnetic fields generated within housing 130 from radiating outside the room in which fMRI system 115 is located.

fMRI system 115 further includes an oxygen tank 135 that is coupled it an interface 140, such as a mask. Oxygen tank 135 and interface 140 are structured to provide the cyclic oxygenation to subject 125 as described herein (see box/step 5 of FIG. 1).

fMRI system 115 also includes a control module 145 that includes all the components that are required to drive the gradient coil system and RF system (for example, a controller, an RF transmitter, an output amplifier, and the like) of fMRI system 115. Control module 145 also includes all the components that are required to acquire the response signals from the body-part (for example, an input amplifier, an Analog-To-Digital Converter, or ADC, and the like). Moreover, control module 145 drives an optional motor (not shown) that is used to move the table 120 to and from the tunnel of hosing 130. Control module 145 is thus structured and configured to generate the signals that are necessary drive fMRI system 115 in order to collect the spatially encoded data, including the training data and non-training data, as described in detail herein.

fMRI system 115 further includes a computer system 150 (for example, a personal computer, or PC), which is coupled to control module 145. Computer system 150 is configured to control fMRI system 115 and to implement the method of FIG. 1 in it various exemplary embodiments as described in detail herein. Computer system 150 thus includes a controller having software for performing such post-processing, including the routines for implementing the various embodiments of the method of generating 4D Oxy-wavelet MRI images as described herein.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A method of non-invasively assessing mitochondrial function in live tissue of a subject, comprising:
providing periodic hypoxia challenges to the subject during a cyclic oxygenation period;
acquiring 4D BOLD fMRI data from the live tissue of the subject during the cyclic oxygenation period; and
analyzing the acquired 4D BOLD fMRI data to determine a measure of mitochondrial function for each of a number of regions of the live tissue.

2. The method according to claim 1, wherein the periodic hypoxia challenges are provided according to an oxygen cycling waveform, wherein the 4D BOLD fMRI data represents a BOLD signal, and wherein the analyzing includes cross-correlating the oxygen cycling waveform with the BOLD signal.

3. The method according to claim 2, wherein the analyzing includes time-frequency analysis of the BOLD signal.

4. The method according to claim 1, wherein the providing periodic hypoxia challenges comprises providing the periodic hypoxia challenges interspersed between cycles of hyperoxia.

5. The method according to claim 1, wherein the acquiring the 4D BOLD fMRI data comprises alternating between collecting a first set of data (D1) and a second set of data (D2), wherein D1 informs temporal basis functions and is collected at a high temporal sampling rate and at a limited sub-Nyquist sampling number of k-space trajectories, and wherein D2 informs spatial coefficient maps and is sparsely sampled (k, t)-space data with extended k-space coverage.

6. The method according to claim 5, wherein the acquiring comprises extracting a number of temporal basis functions from D1 and calculating a number of coefficient images using D2 and the extracted temporal basis functions.

7. The method according to claim 6, wherein the acquiring comprises generating a final 4D image signal by multiplying each of the temporal basis functions by a corresponding one of the coefficient image to create a plurality of products and summing the products.

8. The method according to claim 1, wherein the acquiring comprises generating a final 4D image signal based on the 4D BOLD fMRI data, and wherein the analyzing comprises segmenting the final 4D image signal to create a segmented BOLD waveform.

9. The method according to claim 8, wherein the analyzing further comprises forcing the BOLD waveform to be zero-mean and frequency normalized to create a modified segmented BOLD waveform.

10. The method according to claim 9, wherein the analyzing further comprises determining a BOLD baseline using non-linear curve fitting, and calculating a number of histograms indicative of an amount of the modified segmented BOLD waveform that is above and below the BOLD baseline, wherein the measure of mitochondrial function comprises the number of histograms.

11. The method according to claim 9, wherein the analyzing further comprises down sampling the modified segmented BOLD waveform to create a down sampled BOLD waveform, calculating a BOLD phase prominence based on the down sampled BOLD waveform to localize points of oxygen change, creating a gradient of the BOLD waveform based on the BOLD phase prominence, and determining an average gradient between oxygen states of the cyclic oxygenation using the gradient of the BOLD waveform to calculate a hemodynamic response rate, wherein the measure of mitochondrial function comprises the hemodynamic response rate.

12. The method according to claim 9, wherein the analyzing further comprises cross correlating the modified segmented BOLD waveform with a cyclic oxygenation template of the cyclic oxygenation period, and calculating a scale and directionality of a maximum correlation between the modified segmented BOLD waveform and the cyclic oxygenation template the in a 2-D time-frequency space, and extracting an oxy-wavelet index based on the maximum correlation, wherein the measure of mitochondrial function comprises the oxy-wavelet index.

13. The method according to claim 12, wherein a negative value for the oxy-wavelet index indicates intact mitochondrial function and a positive value for the oxy-wavelet index indicates mitochondrial dysfunction.

14. The method according to claim 13, further comprising using the oxy-wavelet index to create a spatial map.

15. A non-transitory computer readable medium storing one or more programs, including instructions, which when executed by a computer causes the computer to perform the method of claim 1.

16. A system for non-invasively assessing mitochondrial function in live tissue of a subject, comprising:
an fMRI system including a magnet, an RF system and a controller, the controller being structured and configured to:
acquire 4D BOLD fMRI data from the live tissue of the subject during a cyclic oxygenation period wherein periodic hypoxia challenges are experienced by the subject; and
analyze the acquired 4D BOLD fMRI data to determine a measure of mitochondrial function for each of a number of regions of the live tissue.

17. The system according to claim 16, further comprising oxygen source structured to be coupled to the subject for providing the periodic hypoxia challenges to the subject.

18. The system according to claim 16, wherein the periodic hypoxia challenges are provided according to an oxygen cycling waveform, wherein the 4D BOLD fMRI data represents a BOLD signal, and wherein the controller is structured and configured to analyze the acquired 4D BOLD fMRI data by cross-correlating the oxygen cycling waveform with the BOLD signal.

19. The system according to claim 18, wherein the analyzing includes time-frequency analysis of the BOLD signal.

20. The system according to claim 16, wherein the periodic hypoxia challenges are interspersed between cycles of hyperoxia.

21. The system according to claim 16, wherein the acquiring the 4D BOLD fMRI data comprises alternating between collecting a first set of data (D1) and a second set of data (D2), wherein D1 informs temporal basis functions and is collected at a high temporal sampling rate and at a limited sub-Nyquist sampling number of k-space trajectories, and wherein D2 informs spatial coefficient maps and is sparsely sampled (k, t)-space data with extended k-space coverage.

22. The system according to claim 21, wherein the acquiring comprises extracting a number of temporal basis functions from D1 and calculating a number of coefficient images using D2 and the extracted temporal basis functions.

23. The system according to claim 22, wherein the acquiring comprises generating a final 4D image signal by multiplying each of the temporal basis functions by a corresponding one of the coefficient image to create a plurality of products and summing the products.

24. The system according to claim 16, wherein the acquiring comprises generating a final 4D image signal based on the 4D BOLD fMRI data, and wherein the analyzing comprises segmenting the final 4D image signal to create a segmented BOLD waveform.

25. The system according to claim 24, wherein the analyzing further comprises forcing the BOLD waveform to be zero-mean and frequency normalized to create a modified segmented BOLD waveform.

26. The system according to claim 25, wherein the analyzing further comprises determining a BOLD baseline using non-linear curve fitting, and calculating a number of histograms indicative of an amount of the modified segmented BOLD waveform that is above and below the BOLD baseline, wherein the measure of mitochondrial function comprises the number of histograms.

27. The system according to claim 25, wherein the analyzing further comprises down sampling the modified segmented BOLD waveform to create a down sampled BOLD waveform, calculating a BOLD phase prominence based on the down sampled BOLD waveform to localize points of oxygen change, creating a gradient of the BOLD waveform based on the BOLD phase prominence, and determining an average gradient between oxygen states of the cyclic oxygenation using the gradient of the BOLD waveform to calculate a hemodynamic response rate, wherein the measure of mitochondrial function comprises the hemodynamic response rate.

28. The system according to claim 25, wherein the analyzing further comprises cross correlating the modified segmented BOLD waveform with a cyclic oxygenation template of the cyclic oxygenation period, and calculating a scale and directionality of a maximum correlation between the modified segmented BOLD waveform and the cyclic oxygenation template the in a 2-D time-frequency space, and extracting an oxy-wavelet index based on the maximum correlation, wherein the measure of mitochondrial function comprises the oxy-wavelet index.

29. The system according to claim 28, wherein a negative value for the oxy-wavelet index indicates intact mitochondrial function and a positive value for the oxy-wavelet index indicates mitochondrial dysfunction.

30. The system according to claim 29, further comprising using the oxy-wavelet index to create a spatial map.
